(19) United States
(12) Patent Application Publication
(10) Pub. No.: US 2010/0203150 A1
(43) Pub. Date: Aug. 12, 2010

(54) Novel amphiphilic copolymers and fabrication method thereof

(75) Inventors: Yu-Der Lee, Hsin Chu City (TW); Kuo-Yung Chang, Hsin Chu City (TW); Chia-Chun Lin, Hsin Chu City (TW)

Correspondence Address: BACON & THOMAS, PLLC, 625 SLATERS LANE, FOURTH FLOOR, ALEXANDRIA, VA, 22314-1176, US

(73) Assignee: National Tsing Hua University, Hsin Chu City (TW)

(21) Appl. No.: 12/320,843

(22) Filed: Feb. 6, 2009

(51) Int. Cl. A61K 9/14; A61K 31/7072

(52) U.S. Cl. 424/497; 514/50

(57) ABSTRACT

The present invention discloses a comb-like amphiphilic copolymer comprising a hydrophilic segment and a hydrophobic segment. It also discloses a novel fabrication method for synthesizing said amphiphilic copolymers. A novel initiator comprising a drug molecule, e.g. 5′-DFUR, bonded to one or two macromolecules of a hydrophobic polymer is provided. A spherical micelle with core-shell structure is formed by self-aggregation of said amphiphilic copolymer. It discloses a micelle-like nanoparticle comprising one or more said amphiphilic copolymers. The nanoparticles contain said drug molecules innately with no need to encapsulate the desired drug into the nanoparticles. The nanoparticles can serve as micellar drug carriers for delivering the drug. A nanocarrier comprising said nanoparticle and an active water-insoluble substance, e.g. SN-38, Camptothecin, encapsulated in the nanoparticle is also disclosed. The nanocarriers can serve as a means of cocktail therapy to deliver a mixture of two kinds of drugs to affected parts.

### TECHNICAL FIELD

Embodiments of the invention generally relate to the field of high polymer chemistry and, more particularly, to biocompatible and biodegradable amphiphilic copolymers and nanoparticles comprising the same, and a novel fabrication method for synthesizing thereof.

### BACKGROUND

Micelle-like aggregates formed from amphiphilic copolymers have attracted considerable interest in recent years owing to their unique phase behavior in aqueous media and their potential applications as carriers for genes and hydrophobic drugs. Due to their amphiphilic characteristics, block or graft copolymers composed of hydrophilic and hydrophobic segments exhibit surfactant behavior and can form micelles with core-shell structure at critical micelle concentration (CMC). These micelles have a hydrophobic compact inner core and a hydrophilic swollen outer shell in aqueous media, both of which are thermodynamically stable in physiological solution because the hydrophobic blocks in an aqueous phase undergo macromolecular assembly to generate polymeric micelles and micelle-like aggregates. Furthermore, polymer micelles could be easily designed on the nano-scale sizes with a narrow size distribution, which greatly facilitates the regulation of biodistribution.

The hydrophobic segments of the polymeric micelles normally comprise biocompatible and biodegradable materials such as polyesters and their derivatives. Poly(ε-caprolactone) (PCL) is an aliphatic polyester prepared by the ring-opening polymerization (ROP) of ε-caprolactone. PCL has various advantages such as non-toxicity, excellent biodegradability and biocompatibility, and high resistance to water, oil, solvents and chlorine. These unique characteristics of PCL are responsible for its potential medical applications, such as drug carriers in the development of controlled drug delivery systems. Polypeptide block and graft copolymers are also effective copolymers for fabricating drug carriers and can produce core-shell structure with various shapes. Poly(γ-glutamic acid, γ-PGA) is a biosynthetic polypeptide consisting of D- and L-glutamic acid units that are connected by amide linkages between the α-amino and γ-carboxylic groups. Interest in this biomaterial has recently been renewed because of its excellent properties including its water-solubility, biodegradability, biocompatibility, high capacity to absorb water and non-toxicity toward humans and the environment. Recently, several studies have investigated the hydrophilic segments of polymeric micelles, for example, PLLA-block-γ-PGA copolymer (Liang, H. F. et al. Biomaterials 2006, 27, 2051-2059; Liang, H. F. et al. J. Controlled Release 2005, 105, 213-225; Liang, H. F. et al. Bioconjugate Chem. 2006, 17, 291-299) and phenylalanine-graft-γ-PGA copolymer (Matsusaki, M. et al. Chem. Lett. 2004, 33(4), 398-399), because of their high hydrophilic property and excellent water-binding capacity.

The amphiphilic block copolymer disclosed in the U.S. Patent Published Application No. 20080166382 includes one or more hydrophilic polymers, one or more hydrophobic polymer, and one or more zwitterions. This invention also provides a nanoparticle and carrier including the amphiphilic block copolymer for delivery of water insoluble drugs, growth factors, genes, or water insoluble cosmetic substances. The preparation of the biomedical polymer is described as follows. First, a copolymer comprising a hydrophilic block and a hydrophobic block, such as PEG-PCL, PEG-PVL, and PEG-PPL, is prepared. Next, the copolymer is dissolved in a solvent, and its terminal is added with a chemical group to form a modified copolymer. After the modified copolymer is dissolved in a solvent, its modified terminal is reacted with another chemical group, such as trimethylamine (TMA), 1,3-propane sultone (PS), and benzyl histidine, to form zwitterions. Thus, a copolymer comprising a hydrophilic block, a hydrophobic block, and zwitterions is obtained.

The aim of this invention was to investigate the synthesis, characterization and properties of novel comic-like amphiphilic copolymers. These novel amphiphilic copolymers are capable of forming polymeric micelles in the aqueous solution and their physicochemical properties are discussed herein.

### SUMMARY OF THE INVENTION

It is one object of the present invention to provide a comb-like amphiphilic copolymer comprising a hydrophilic segment and a hydrophobic segment.

It is another object of the present invention to disclose a novel fabrication method different from the conventional ones for synthesizing said comb-like amphiphilic copolymers, the method comprising: synthesizing an initiator comprising a drug molecule bonded to one or two macromolecules of a hydrophobic polymer; and grafting said initiators onto a pre-polymerized hydrophilic segment to form a comb-like amphiphilic copolymer.

It is also another object of the present invention to disclose the novel initiator comprising the drug molecule bonded to one or two macromolecules of the hydrophobic polymer for synthesizing said comb-like amphiphilic copolymers through said novel fabrication method.

It is also another object of the present invention to provide a biodegradable or biocompatible amphiphilic copolymer.

It is another object of the present invention to provide a spherical micelle with core-shell structure formed by self-aggregation of said amphiphilic copolymer in the aqueous phase.

It is also another object of the present invention to provide a micelle-like nanoparticle comprising one or more said amphiphilic copolymers in aqueous solution.

It is still another object of the present invention to disclose said nanoparticle carrying said drug molecule innately with no need to encapsulate the desired drug into the nanoparticle. The nanoparticles can serve as micellar drug carriers for delivering the drug to affected body parts.

It is still another object of the present invention to provide said amphiphilic copolymer further comprising one or more selectivity-providing molecules functioning as biomarker(s) bonded to said amphiphilic copolymer to enable said drug carrier to target the affected tissues with little harm toward normal tissues.

It is still another object of the present invention to disclose a nanocarrier comprising said nanoparticle and a bioactive water-insoluble or hydrophobic substance encapsulated in said nanoparticle. The nanocarriers are able to serve as a means of cocktail therapy to deliver a mixture of two kinds of drugs to affected tissues.

A detailed description is given in the following embodiments with reference to the accompanying drawings.

### BRIEF DESCRIPTION OF THE DRAWINGS

Embodiments of the invention are illustrated by way of example, not by way of limitation, and the following figures and table relate particularly to a preferred embodiment of the present invention, the nanoparticles composed of 5′-DFUR-PCL-γ-PGA comb-like amphiphilic copolymers.

FIG. 1 (A) shows the synthesis of 5′-DFUR-PCL polymer;

FIG. 1 (B) shows the synthesis of 5′-DFUR-PCL-γ-PGA copolymer;

FIG. 2 shows the flowchart of the fabrication method for synthesizing comb-like amphiphilic copolymers;

FIG. 3 (A) shows ¹H NMR spectrum of 5′-DFUR;

FIG. 3 (B) shows ¹H NMR spectrum of 5′-DFUR-PCL polymer;

FIG. 3 (C) shows the partial view of FIG. 3 (B), δ 7.3-9.0; and

FIG. 3 (D) shows the partial view of FIG. 3 (B), δ 4.0-6.0;

FIG. 4 shows ¹H NMR spectrum of 5′-DFUR-PCL-γ-PGA copolymer, * the signals of N-acylurea;

FIG. 5 shows FTIR spectra of (A) γ-PGA(Na); (B) γ-PGA(H); (C) 5′-DFUR-PCL polymers; and (D) 5′-DFUR-PCL-γ-PGA copolymers;

FIG. 6 shows different water contact angles of 5′-DFUR-PCL-γ-PGA copolymers with various γ-PGA contents;

FIG. 7 shows DSC thermograms of various 5′-DFUR-PCL-γ-PGA copolymers: (A) γ-PGA(H); (B) 5′-DFUR-PCL-γ-PGA₈₆; (C) 5′-DFUR-PCL-γ-PGA₂₂; and (D) 5′-DFUR-PCL;

FIG. 8 shows excitation spectra (λₑₓ=390 nm) of pyrene (1.2×10⁻⁶ M) recorded in the presence of increasing concentrations of 5′-DFUR-PCL-γ-PGA₆₄ copolymer from 0.02 to 167 mg/L;

FIG. 9 shows the relationship of the intensity ratio (I₃₃₈/I₃₃₅) to the 5′-DFUR-PCL-γ-PGA copolymer concentration;

FIG. 10 shows zeta potential measurements of various 5′-DFUR-PCL-γ-PGA copolymers: (A) 5′-DFUR-PCL-γ-PGA₈₆; (B) 5′-DFUR-PCL-γ-PGA₇₁; (C) 5′-DFUR-PCL-γ-PGA₆₄; and (D) 5′-DFUR-PCL-γ-PGA₂₂; and

FIG. 11 shows TEM images of the 5′-DFUR-PCL-γ-PGA copolymers: (A) 5′-DFUR-PCL-γ-PGA₈₆; (B, E) 5′-DFUR-PCL-γ-PGA₇₁; (C) 5′-DFUR-PCL-γ-PGA₆₄; and (D, F) 5′-DFUR-PCL-γ-PGA₂₂.

### DETAILED DESCRIPTION

The following description is of the best-contemplated mode of carrying out the invention. This description is made for the purpose of illustrating the general principles of the invention and should not be taken in a limiting sense. The scope of the invention is best determined by reference to the appended claims.

One embodiment of the invention provides a comb-like amphiphilic copolymer comprising a hydrophilic segment, e.g. γ-PGA (poly(γ-glutamic acid)), and a hydrophobic segment, e.g. 5′-DFUR-PCL (5′-deoxy-5-fluorouridine-poly(ε-caprolactone)).

The copolymer is a comic-like amphiphilic copolymer. The copolymer with a CMC (Critical Micelle Concentration) of about 1.0-10.0 mg/L comprises a backbone formed by the hydrophilic chain (e.g. γ-PGA), having projections like the teeth of a comb formed by hydrophobic side chains (e.g. 5′-deoxy-5-fluorouridine-poly(ε-caprolactone); i.e. 5′-DFUR-PCL). The hydrophobic segment contains all these hydrophobic side chains. The hydrophilic segment has a molecular weight of about 500-35,000 and may include a hydrophilic polymer such as poly-γ-glutamic acid (γ-PGA), polyethylene glycol (PEG), starch, chondroitin sulfate, chitosan, dextran or hyaluronic acid (HA). The hydrophobic segment, comprising a plurality of drug molecules and a plurality of hydrophobic polymers, has a molecular weight of about 500-35,000. Furthermore, each of the plurality of drug molecules is bonded to one or two macromolecules of the hydrophobic polymer. The hydrophobic polymer portion (drug molecules excluded) of the hydrophobic segment may include a polyester, such as polycaprolactone (PCL), polyvalerolactone (PVL), poly(lactic-co-glycolic acid) (PLGA), polylactic acid (PLA), polybutyrolactone (PBL), polyglycolide, or polypropiolactone (PPL). The amphiphilic copolymer is biodegradable and biocompatible.

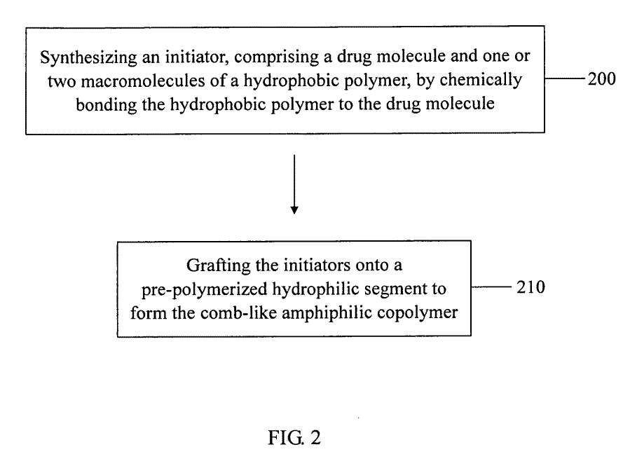

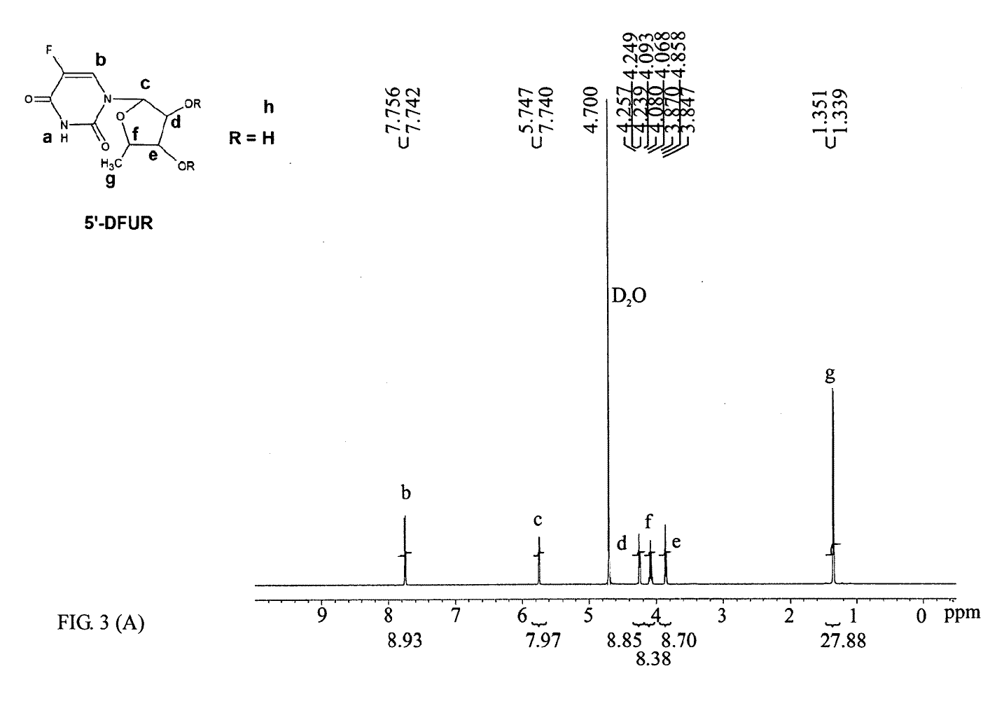

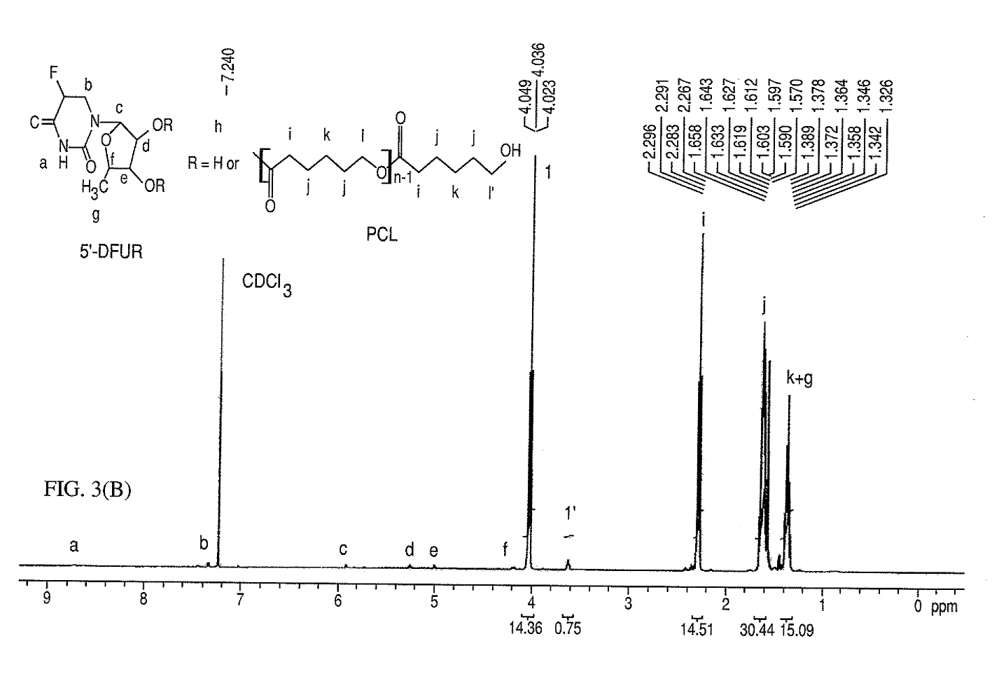

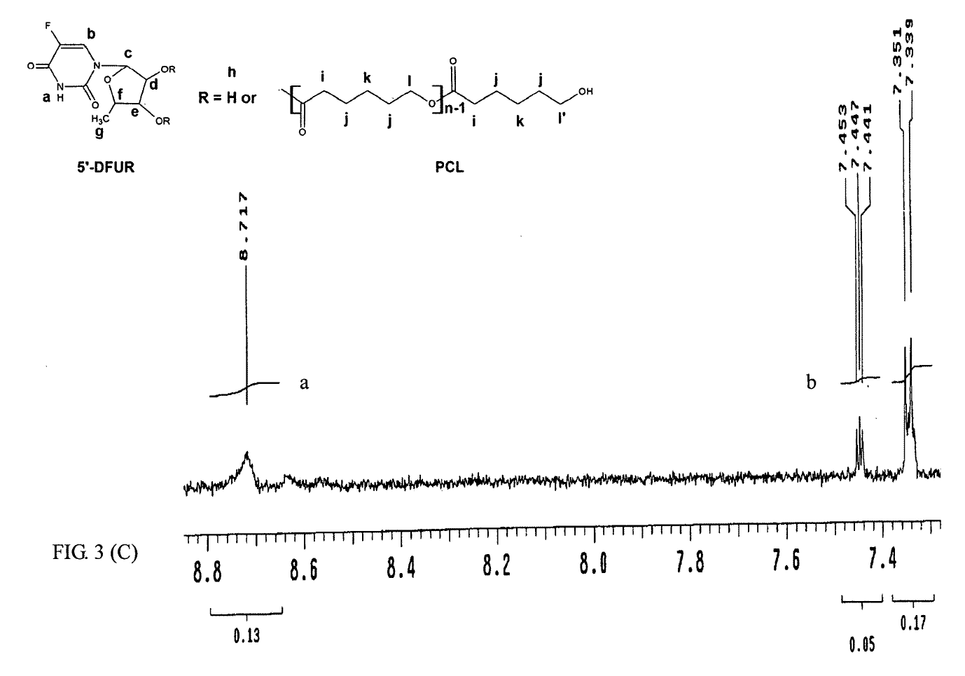

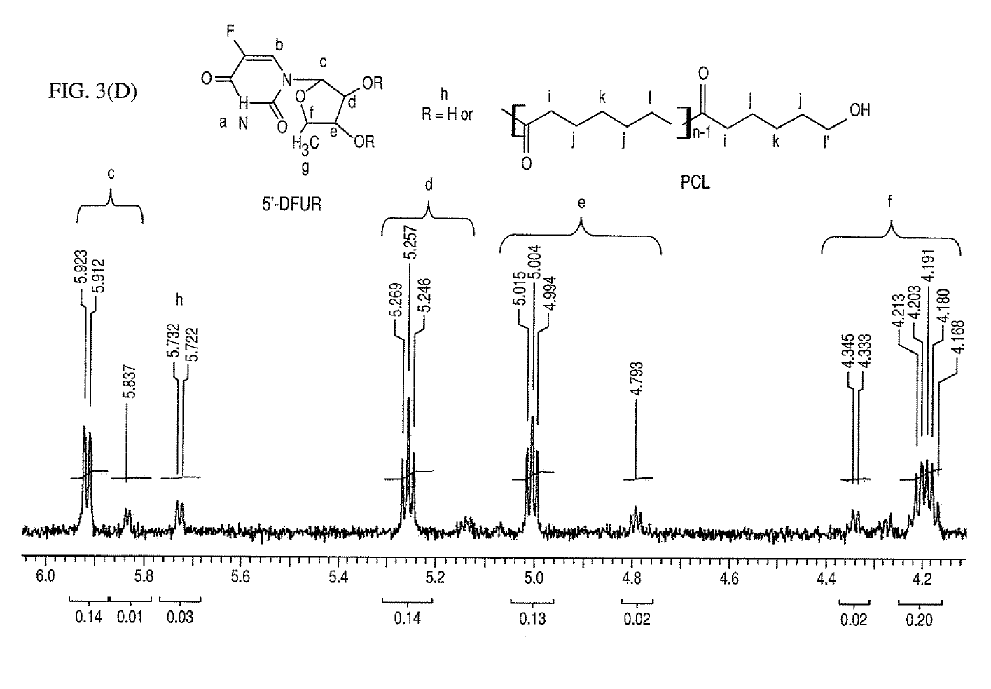

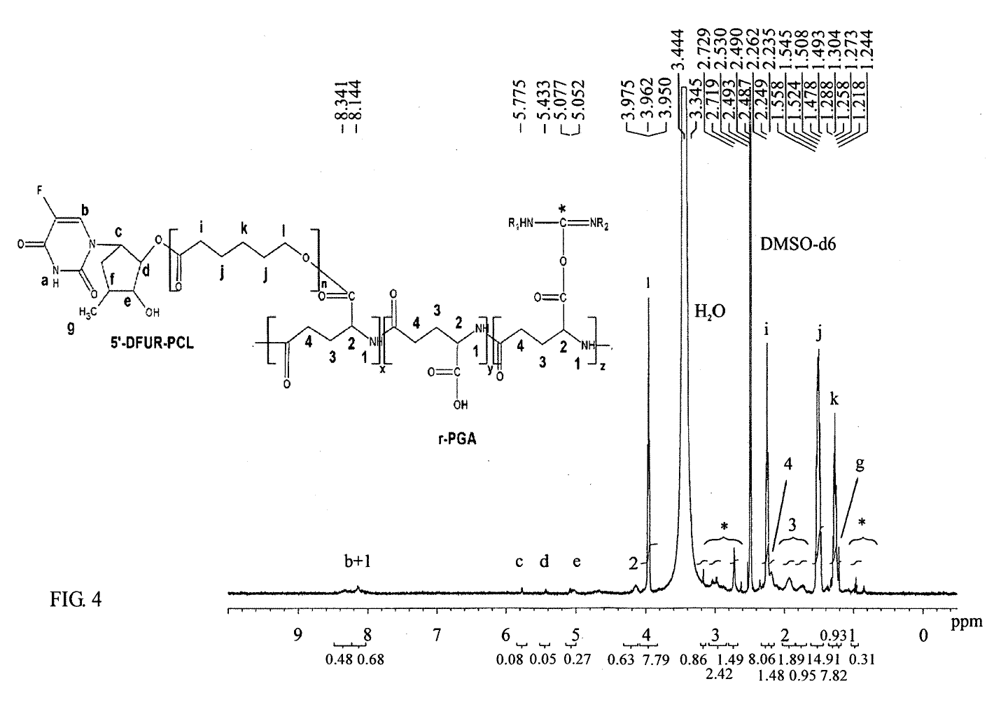

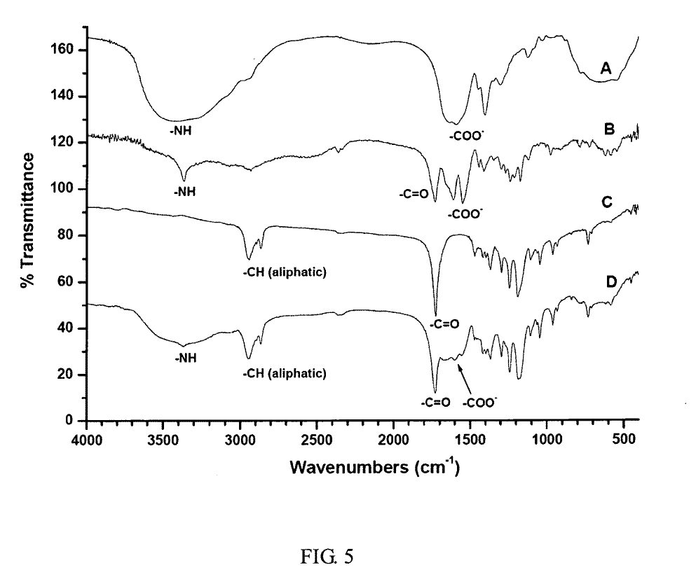

Referring to the FIG. 2, it shows the flowchart of the fabrication method for synthesizing the comb-like amphiphilic copolymers. In step 200, an initiator, comprising a drug molecule and one or two macromolecules of a hydrophobic polymer, was synthesized by chemically bonding the hydrophobic polymer to the drug molecule. In step 210, the comb-like amphiphilic copolymer was synthesized by grafting the initiators onto a pre-polymerized hydrophilic segment. In an embodiment, a novel initiator, 5′-DFUR-PCL polymer, was synthesized by the ROP (Ring-Opening Polymerization) of ε-caprolactone (ε-CL), initiated by antitumor agent Doxifluridine (5′-DFUR). The hydrophobic 5′-DFUR-PCL polymer was then grafted to hydrophilic γ-PGA polymer forming a 5′-DFUR-PCL-γ-PGA comb-like amphiphilic copolymer. These novel amphiphilic copolymers are capable of forming self-assembled polymeric micelles in the aqueous solution and their physicochemical properties are discussed in detail in a later part.

In a preferred embodiment, the polymeric micelles formed by 5′-DFUR-PCL-γ-PGA amphiphilic copolymers were prepared by nanoprecipitation method (Hu, Y. et al. Biomaterials 2003, 24, 2395-2404). In some embodiments, the invention provides a micelle-like self-aggregated spherical nanoparticle comprising one or more said amphiphilic copolymers. In one embodiment, the micelles are composed of a hydrophilic shell of γ-PGA and a hydrophobic core of 5′-DFUR-PCL. This well-defined core-shell structure also proved that these 5′-DFUR-PCL-γ-PGA amphiphilic copolymers could form micelle-like nanoparticles in aqueous solution. The nanoparticle has a hydrophobic interior with the drug molecule therein and a hydrophilic surface and its diameter is about 20-800 nm.

Because of the chain flexibility of the hydrophilic segments in blood, and the decomposability of the hydrophobic segments by enzymes or hydrolysis of the nanoparticles, these novel biomedical nanoparticles are able to serve as biocompatible, biodegradable, and invisible to immune cells drug carriers. After the hydrophobic polymer is decomposed, remaining harmless substances such as the hydrophilic segment are dissolved in blood and then removed from the renal system.

In one embodiment, the hydrophobic segment may be 5′-DFUR-PCL, thus the nanoparticles being nanocarriers of the 5′-DFUR (Doxifluridine). This drug delivery system will prolong in vivo drug actions as extended-release technology does, decrease drug metabolism, and reduce drug toxicity toward normal tissues. Above mentioned may improve the delivery and the effectiveness of antitumor drugs. In some embodiments, carriers can also be used in the targeted drug delivery, for example, marking the polymer by one or more selectivity-providing molecules functioning as biomarker(s), e.g. folic acid, to increase the effectiveness of drug delivery to the target sites of pharmacological actions. The nanocarriers are preferably be delivered by, but not limited to, injection, and routes of administration may include topical routes, such as inhalational (pneumonial) or nasal, enteral routes, such as oral or rectal, parenteral routes, such as injection or infusion, or other parenteral routes, such as transdermal or transmucosal.

In some embodiments, the initiators for syntheses of the hydrophobic segments may include 5′-DFUR (Doxifluridine), 5′-DFCR, Capecitabine (Xeloda®), other nucleotide analogs, other precursor analogs, or other antimetabolites.

The amphiphilic polymeric micelles formed therefrom carry the antitumor drug natively, for example, 5′-DFUR, 5′-DFCR, and Capecitabine, because these antitumor drugs are used as the initiators of the hydrophobic polymer syntheses. In this way, there is no need to encapsulate desired drugs into micelles, and this is the most significant difference from the prior art. The novel polymerization method disclosed herein for synthesizing the copolymers is more economical, which may cut the steps of the preparation process and reduce the prime cost, particularly saving steps of the common process for grafting the drug molecules onto the polymers and saving the general process for encapsulating desired drugs into micelles. Furthermore, it may replace the present techniques of the polymer syntheses. The above-mentioned micelle-like self-assembled nanoparticles are able to serve as drug carriers with chemotherapeutic agent, Doxifluridine (5′-DFUR), for instance, to deliver this agent to cancerous tissues. In this embodiment, 5′-deoxy-5-fluorouridine (5′-DFUR) is enzymatically converted to 5-fluorouracil (5-FU) in the tumor, where it inhibits DNA synthesis and slows growth of tumor tissue.

In some other preferred embodiments, the nano-micelle carriers are designed from the concept of the cocktail therapy/combination therapy, in which there are more than one kind of chemotherapeutic agents administered, for example, 5′-DFUR and SN-38 (an inhibitor of topoisomerase I), 5′-DFCR and CPT-11 (Irinotecan, a topoisomerase I inhibitor), Capecitabine and Docetaxel (Taxotere®, a mitotic inhibitor), or the combination thereof. The hydrophobic nature of the core of the micelles enables water-insoluble compounds to be encapsulated into the self-assembly micelles in the aqueous solutions. This will serve the micelle as a nanocarrier of cocktail therapy characteristic comprising two types of antineoplastic agents with different mechanisms of action, for example. In one preferred embodiment, 5′-DFUR-PCL-γ-PGA copolymer micelles encapsulating SN-38 may provide a way to cure the colorectal cancers effectively. In another preferred embodiment, Capecitabine-PCL-γ-PGA copolymer micelles containing docetaxel may be used in the treatment of metastatic breast and colorectal cancers. In treating some cancers, even drug-resistant colorectal cancers, this kind of sustained-release (SR) or timed-release cocktail therapy will dramatically enhance the efficacy of both two drugs, for example, 5-FU and CPT-11 (Guichard, S. et al. Biochem. Pharmacol. 1998, 55, 667-676).

In some embodiments of the invention, the water-insoluble or hydrophobic compounds that can be encapsulated into the self-assembly micelles include CPT-11 (Irinotecan/Campto®), SN-38, Camptothecin (CPT) and its derivatives, and any other water-insoluble or hydrophobic anticancer agents. The disclosed nanoparticles functioning as nanocarriers herein allow active ingredients to release at cancerous tissues over time and to keep steadier levels of the drugs in the bloodstream, hence enabling both of antitumor drugs to be more efficacious. Moreover, the described nanocarriers are able to decrease adverse effects resulting from the chemotherapy and to prolong half-lives of the anticancer agents.

In some embodiments of the invention, the cancers or tumors can be treated including, but not limited to, stomach cancer, colon cancer, colorectal cancer, breast cancer, lung cancer, esophageal cancer, head and neck squamous cell carcinomas (HNSCC's), primary hepatocellular carcinoma (HCC) or metastatic liver cancer, pancreatic cancer, bile duct cancer, gallbladder cancer, small cell lung carcinoma, non-small cell lung carcinoma, cervical cancer, ovarian cancer, lymphoma, etc.

In some embodiments, thermal analyses reveal that copolymers have lower melting points than their corresponding homopolymers. Varying the proportion of the hydrophilic segment in the amphiphilic copolymers would change their hydrophilic abilities, and the preferable range of the proportion of the hydrophilic segment in the copolymers is around 20-90%. Furthermore, the average sizes of the micelles could be changed by varying the graft copolymer composition thereof. The diameter of the nanoparticles formed therefrom is about 20-800 nm.

### Example

The preferred embodiment of the present invention described below relate particularly to the preparation of nanoparticles composed of 5′-DFUR-PCL-γ-PGA comb-like amphiphilic copolymers that may further comprise a water-insoluble or hydrophobic biomedical compound to serve as nanocarriers for drug delivery. While the description is set forth in specific details of the preferred embodiment, it will be appreciated that the description is illustrative only and should not be construed in any way to limit the scope of the invention. Moreover, it is obvious to those skilled in the art that various applications of the invention and modifications or equivalent changes thereto are also encompassed within the general concepts described as follows.

### Synthesis of 5′-DFUR-PCL

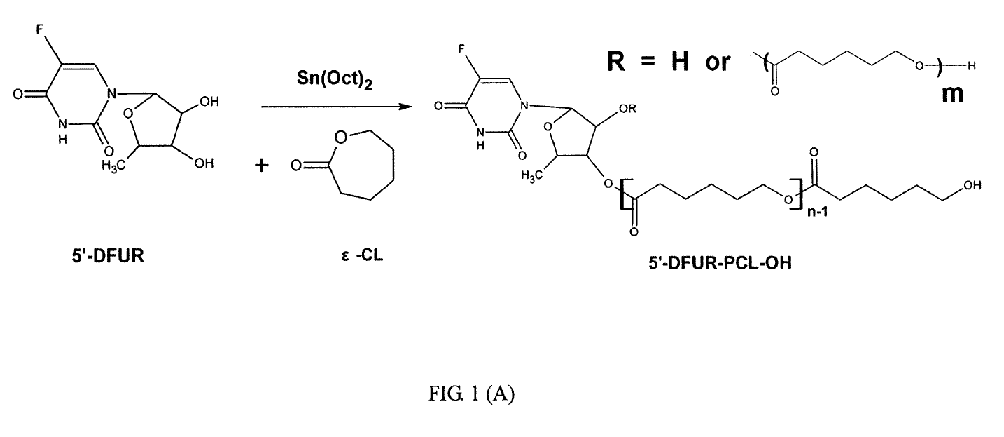

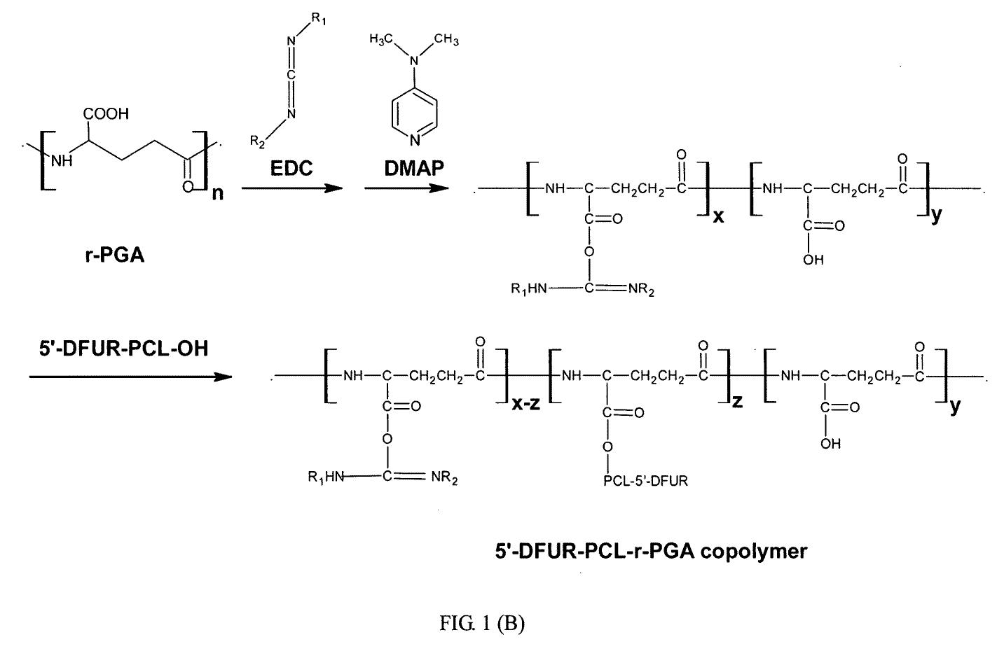

In a preferred embodiment, the synthesis of 5′-DFUR-PCL was synthesized from antitumor agent Doxifluridine (5′-DFUR) by the ring opening polymerization of c-caprolactone (ε-CL) using tin(II) 2-ethylhexanoate (Sn(Oct)₂) as the catalyst (Chang, K. Y. et al. Acta Biomaterialia 2008). Briefly, Sn(Oct)₂ solution (0.5% w/w of ε-CL) was accurately weighed and placed in a dried glass flask, then evaporated under vacuum at room temperature for 30 min to remove all of the hexane completely. After 5′-DFUR (0.369 g) and ε-CL (5.479 g) had been added and mixed to homogeneity, the flask was exhausted under vacuum for degassing and purged with dry nitrogen three times. Finally, 5′-DFUR reacted with ε-CL in the presence of Sn(Oct)₂ as the catalyst at 140° C. for 24 h under nitrogen. After polymerization, the crude product was cooled to room temperature, dissolved in THF, and then precipitated into excess deionized water twice to remove any unreacted 5′-DFUR. The obtained product was further purified with cold ethyl ether and dried under vacuum. FIG. 1 (A) presents the method for preparing 5′-DFUR-PCL polymer.

### Synthesis of 5′-DFUR-PCL-γ-PGA Copolymers

In a preferred embodiment, the synthesis of 5′-DFUR-PCL-γ-PGA copolymers can be carried out as follows. γ-PGA(Na) was converted to γ-PGA(H) in a HCl solution and was adjusted to a pH value of 2.0 (Kunioka, M. et al. J. Appl. Polym. Sci. 1997, 65(10), 1889-1896). The carboxylic group of γ-PGA(H) was activated by EDC and then 5′-DFUR-PCL was grafted to γ-PGA(H) (Murakami, S. et al. Biomacromolecules 2006, 7, 2122-2127). Briefly, γ-PGA(H) (0.1 g) was dissolved in DMSO (9 mL) by ultrasonication and DMAP (0.095 g) was subsequently added to the solution. After predetermined amounts of 5′-DFUR-PCL had been added and dissolved completely, the homogenous solution was mixed with 1 mL of EDC solution (0.148 g, in DMSO) at 40° C. in a nitrogen atmosphere and the reaction was allowed to continue for 24 h. It was then dialyzed with a membrane (spectrum, MWCO: 12,000-14,000) for 3 days to remove all the unreacted γ-PGA(H) and was lyophilized until dry. The obtained products were further purified twice in an excess of acetone to remove the un-grafted 5′-DFUR-PCL. The final products were collected by centrifugation and dried in vacuum at room temperature for 24 h. FIG. 1 (B) presents the procedures for preparing 5′-DFUR-PCL-γ-PGA copolymer. In some embodiments, it showed that the recovery yield and the content of γ-PGA in 5′-DFUR-PCL-γ-PGA declined as the feed ratios (5′-DFUR-PCL/γ-PGA) increased from 1 to 15. The proportions of γ-PGA in the 5′-DFUR-PCL-γ-PGA copolymers were 86%, 71%, 62% and 22% with respect to different feed ratios, respectively. In this work, EDC played an important role to activate the carboxylic group of γ-PGA, which then reacted with the hydroxyl group of 5′-DFUR-PCL to generate comb-like 5′-DFUR-PCL-γ-PGA copolymer.

### Preparation of Polymeric Micelles

In some embodiments, polymeric micelles were prepared by nanoprecipitation method (Hu, Y. et al. Biomaterials 2003, 24, 2395-2404). 5′-DFUR-PCL-γ-PGA copolymer (10 mg) was dispersed in acetone (2 mL) by ultrasonication. The obtained organic solution was added dropwise into stirring deionized water (10 mL) at room temperature and then dispersed by ultrasonication again. Subsequently, acetone was removed by reducing the pressure at room temperature for 1 h. Finally, the resulting aqueous solution was filtered through a 0.45-μm filter membrane to collect polymeric micelles.

### Solubility and Water Contact Angle of 5′-DFUR-PCL-γ-PGA Copolymer

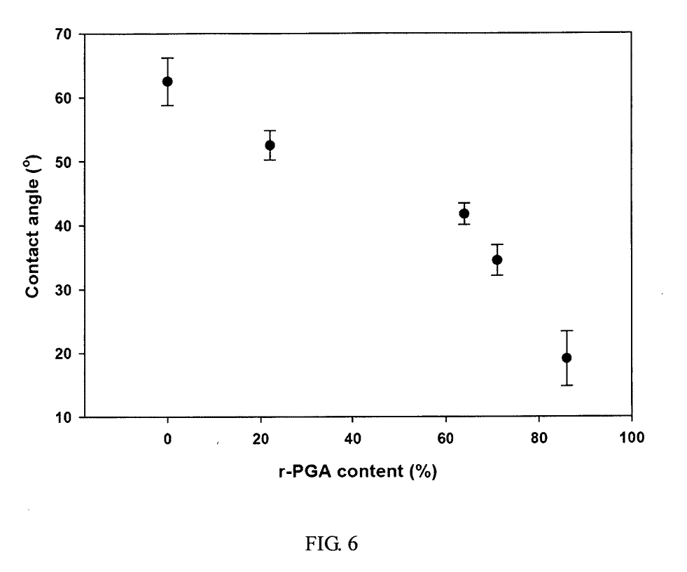

γ-PGA(H), 5′-DFUR-PCL and 5′-DFUR-PCL-γ-PGA copolymers are soluble in some common organic solvents and co-solvents. It reveals that the grafted copolymers are soluble in the co-solvent, but insoluble in most aqueous or organic solvents such as acetone, THF, chloroform, dimethylchloride, DMF and DMSO. It is probably due to the amphiphilic properties of the 5′-DFUR-PCL-γ-PGA copolymer, which inhibits its dispersion and dissolution in common solvents. The solubility of the 5′-DFUR-PCL-γ-PGA in DMSO will be improved if small amount of HCl is introduced so as to convert COO⁻ to COOH in the γ-PGA segments. The wetting behavior of coated glass surfaces containing various amounts of 5′-DFUR-PCL-γ-PGA copolymers was investigated by measuring water contact angle (FIG. 6). The water contact angle of pristine 5′-DFUR-PCL film was 62.5° while those of the 5′-DFUR-PCL-γ-PGA copolymers could be reduced to 19° at 86% γ-PGA content. Furthermore, the water contact angle increased with decreasing γ-PGA content. This strongly suggests that varying the amount of γ-PGA in 5′-DFUR-PCL-γ-PGA copolymer can change hydrophilicity of the copolymer.

### Analyses of Thermal Properties

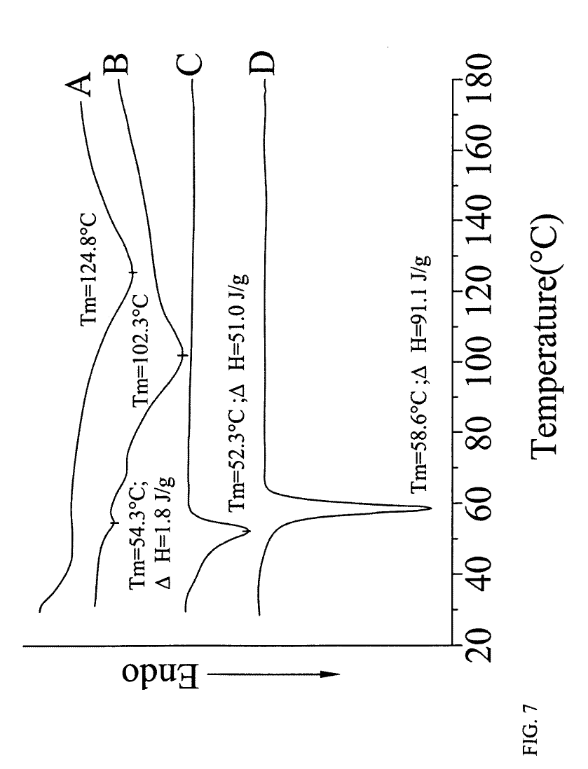

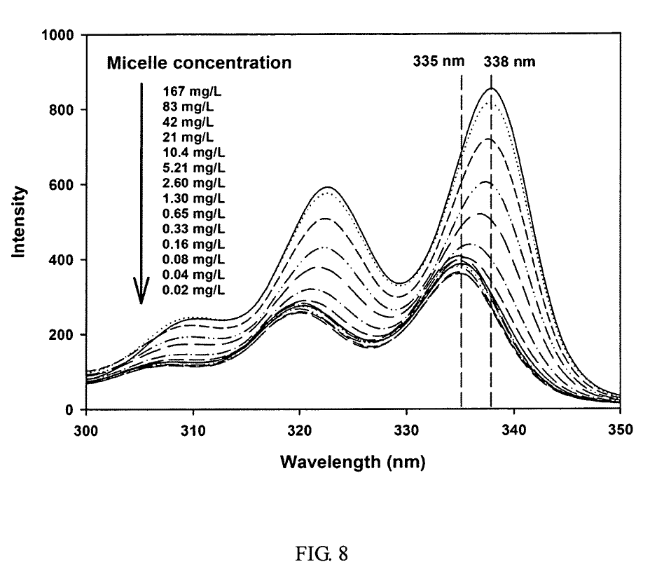

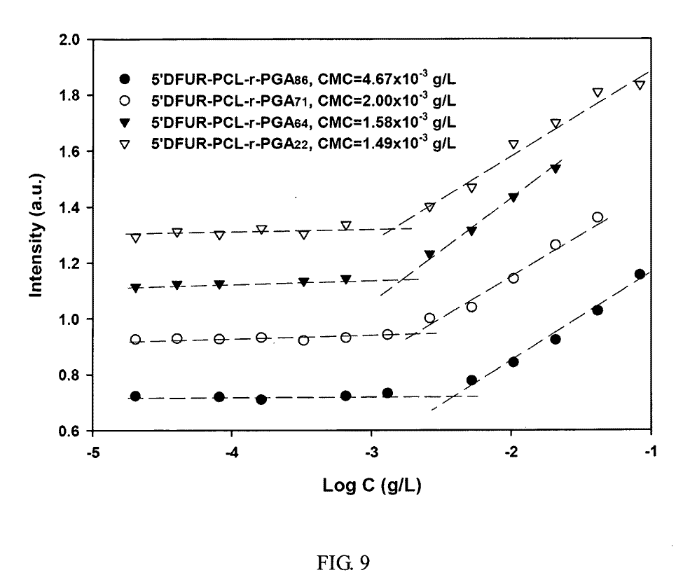

The melting behaviors of the 5′-DFUR-PCL-γ-PGA copolymers were investigated by DSC as shown in FIG. 7. 5′-DFUR-PCL is a semi-crystalline polyester and its melting transition temperature (Tₘ) is approximately 59° C. (FIG. 7 (D)) while γ-PGA is a poly amino acid with a broad endothermal peak (FIG. 7 (A)). Two endothermic peaks were observed during the heating process of 5′-DFUR-PCL-γ-PGA₈₆ (FIG. 7 (B)) and the Tₘ values of γ-PGA and 5′-DFUR-PCL segments in this copolymer were both lower than their corresponding homopolymers (FIGS. 7 (A) and (D)). The introduction of 5′-DFUR-PCL into the copolymer causes the change of crystalline morphology of the γ-PGA and resulting in the decrease of its Tₘ value. Additionally, the endothermic peak of γ-PGA disappeared during the heating of 5′-DFUR-PCL-γ-PGA₂₂ (FIG. 7 (C)). The DSC measurements suggested that the melting endotherm and enthalpy of fusion in γ-PGA and 5′-DFUR-PCL segments of the 5′-DFUR-PCL-γ-PGA copolymers affected each other strongly (Rong, G. et al. Biomacromolecules 2003, 4, 1800-1804; Xie, W. et al. Polymer 2007, 48, 6791-6798).

### Critical Micelle Concentration (CMC)

The formation of micelles was monitored by examining the photophysics of pyrene, a hydrophobic probe that reveals changes in its microenvironment by the changes in its fine emission structure (Zhao, C. et al. Langmuir 1990, 6, 514-516). Pyrene strongly fluoresces in a non-polar environment; however, it has weak fluorescence intensity in a polar environment such as water. This method is based on the difference between the fluorescence spectrum of pyrene in water and that of pyrene in the hydrophobic core of polymeric micelles (Kwon, G. et al. Langmuir 1993, 9, 945-949; Astafieva, I. et al. Macromolecules 1993, 26, 7339-7352). In some embodiments, the CMC (Critical Micelle Concentration) of the amphiphilic copolymers is about 1.0-10.0 mg/L.

### Size and Zeta Potential of Micelles

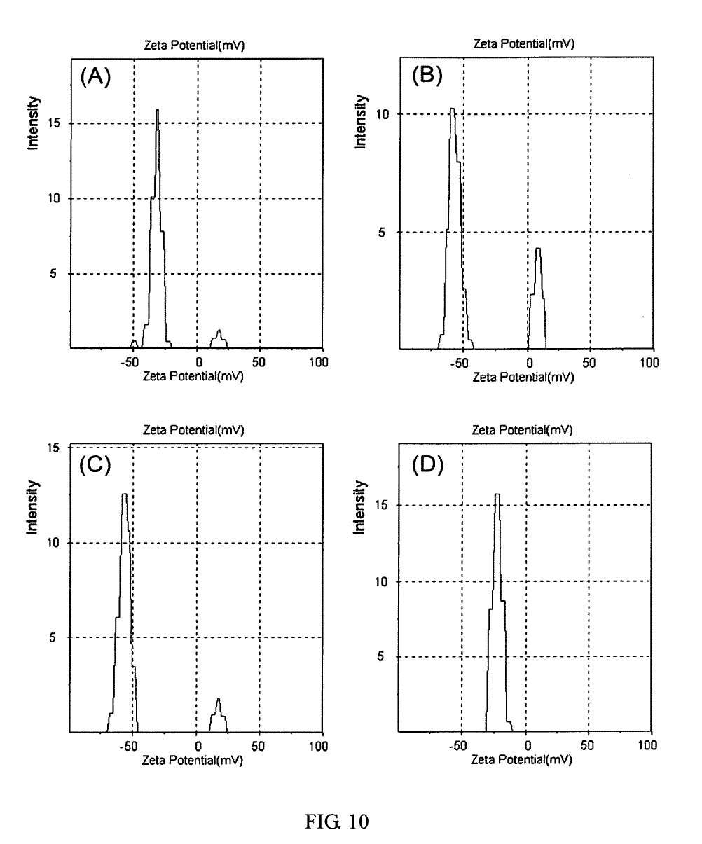

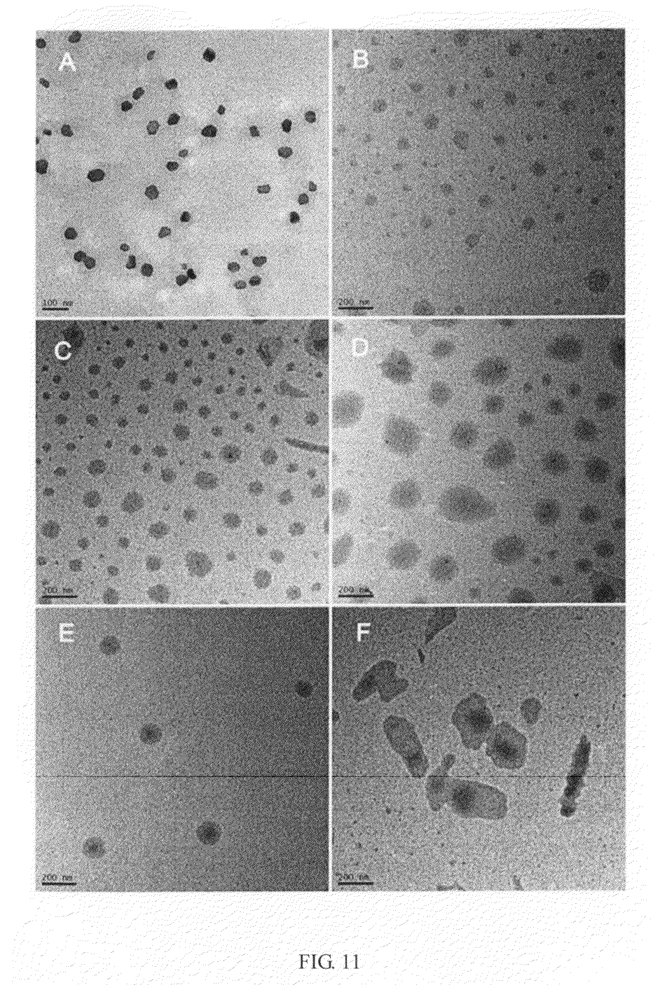

5′-DFUR-PCL-γ-PGA micelles were prepared by a precipitation method. 5′-DFUR-PCL-γ-PGA copolymer is able to form micelles because of its amphiphilic characteristics. The particle size, size distribution and surface charge of the γ-PGA micelles in deionized water were measured by DLS (Dynamic Light Scattering) and zeta potential (electrokinetic potential) measurements, as shown in Table 1. In this embodiment, the mean diameters of the 5′-DFUR-PCL-γ-PGA micelles were increased from 130 to 230 nm with increasing feed ratio (5′-DFUR-PCL/γ-PGA) of the copolymer at constant γ-PGA content. These findings suggest that the hydrophobic segments in the core determine the size of the 5′-DFUR-PCL-γ-PGA micelles (Lee, C. T. et al. Biomacromolecules 2006, 7, 1179-1186). Therefore, the mean diameters of 5′-DFUR-PCL-γ-PGA micelles increase with the increasing 5′-DFUR-PCL content. The 5′-DFUR-PCL-γ-PGA micelles had a highly negative charge in deionized water due to the carboxyl groups in γ-PGA (Table 1). The zeta potentials of 5′-DFUR-PCL-γ-PGA micelles were −28.2±2.6 mV, −21.6±4.0 mV, −20.1±3.9 mV and −16.7±1.9 mV respectively. However, some 5′-DFUR-PCL-γ-PGA copolymers may show positive charges due to residuals of unactiviated EDC (FIG. 10 (A)-10 (C)). The γ-PGA content in 5′-DFUR-PCL-γ-PGA₈₆ is high so that its surface negative charges exceed those of other copolymers. On the other hand, 5% DFUR-PCL-γ-PGA₂₂ with more grafted 5′-DFUR-PCL has small amount of residual unactiviated EDC and so less surface negative charges and no positive charges (FIG. 10 (D)). In some other embodiments of the invention, the diameter of the micelles is around 20-800 nm. In some embodiments, the range of zeta potential is nearly −70-10 mV.

### Morphology of γ-PGA-5′-DFUR-PCL Micelles

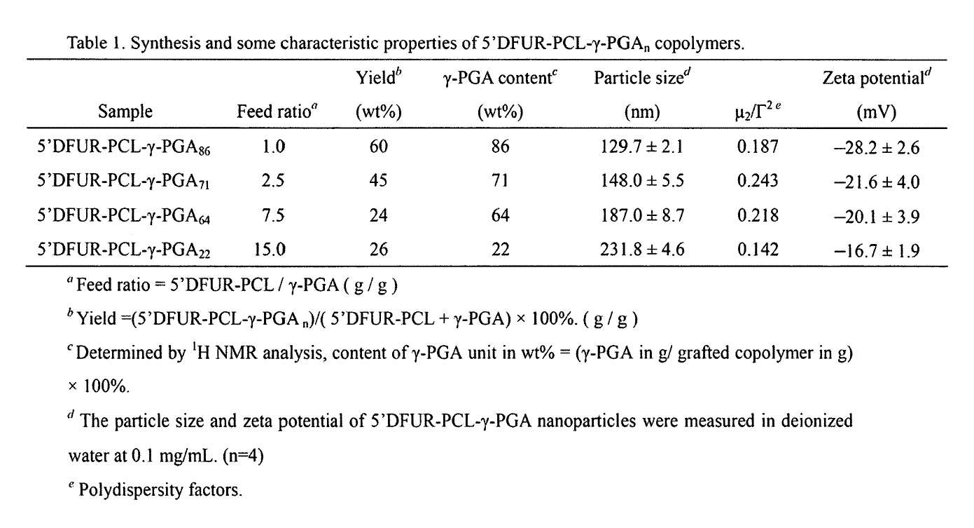

TEM was adopted to visualize directly the morphology, characteristics and the size of the micelles. TEM photographs demonstrate that the copolymer micelles, except the 5′-DFUR-PCL-γ-PGA₂₂ micelles are spherical (FIG. 11). FIGS. 11 (E) and 11 (F) also clearly exhibits micelles with a shell of γ-PGA and a core of 5′-DFUR-PCL. This well-defined core-shell structure also proved that these 5′-DFUR-PCL-γ-PGA copolymers could form micelle-like nanoparticles in aqueous solution. However, the particle size estimated from the TEM images is slightly smaller than those determined by DLS analysis. This may be due to that the DLS analysis reveals only the hydrodynamic diameter upon swelling in aqueous solution, whereas TEM reveals the diameter of dry micelles (Lee, J. et al. J. Controlled Release 2004, 94, 323-335). Additionally, elliptical morphologies (FIG. 11 (F)) of the 5′-DFUR-PCL-γ-PGA₂₂ micelles were formed as the content of 5′-DFUR-PCL increased, perhaps because of the more amount of the 5′-DFUR-PCL in core of the micelles and the higher the hydrophobicity of the 5′-DFUR-PCL (Zhou, Z. et al. J. Am. Chem. Soc. 2003, 125, 10182-10183).

What is claimed is:

1. An amphiphilic copolymer, comprising a hydrophilic segment and a hydrophobic segment grafted to said hydrophilic segment, wherein the shape of said amphiphilic copolymer includes a comb-like shape.

2. The amphiphilic copolymer of claim 1, wherein said hydrophobic segment comprises a plurality of drug molecules and a plurality of hydrophobic polymers.

3. The amphiphilic copolymer of claim 2, wherein each of said plurality of drug molecules is bonded to one or two macromolecules of said hydrophobic polymer.

4. The amphiphilic copolymer of claim 2, wherein said plurality of drug molecules of said hydrophobic segment include 5′-DFUR (Doxifluridine), 5′-DFCR, Capecitabine, nucleotide analogs, precursor analogs, or antimetabolites.

5. The amphiphilic copolymer of claim 2, wherein said plurality of hydrophobic polymers of said hydrophobic segment include polyesters.

6. The amphiphilic copolymer of claim 2, wherein said plurality of hydrophobic polymers of said hydrophobic segment include polycaprolactone (PCL), polyvalerolactone (PVL), poly(lactic-co-glycolic acid) (PLGA), polylactic acid (PLA), polybutyrolactone (PBL), polyglycolide, and polypropiolactone (PPL).

7. The amphiphilic copolymer of claim 2, wherein a spherical micelle-like nanoparticle with core-shell structure is formed by self-assembly of said amphiphilic copolymer, wherein said nanoparticle has a hydrophobic interior with said drug molecules therein and a hydrophilic shell/surface.

8. The amphiphilic copolymer of claim 1, wherein said hydrophobic segment has a molecular weight of about 500-35,000.

9. The amphiphilic copolymer of claim 1, wherein said hydrophilic segment has a molecular weight of about 500-35,000.

10. The amphiphilic copolymer of claim 9, wherein said hydrophilic segment includes poly-γ-glutamic acid (γ-PGA), polyethylene glycol (PEG), starch, chondroitin sulfate, chitosan, dextran or hyaluronic acid (HA).

11. The amphiphilic copolymer of claim 1, wherein the range of the proportion of said hydrophilic segment in said amphiphilic copolymer is about 20-90%.

12. The amphiphilic copolymer of claim 1, wherein said copolymer is biodegradable or biocompatible.

13. A nanocarrier delivered to affected body parts, comprising:
a nanoparticle, comprising one or more amphiphilic copolymers; and
drug molecules, bonded to hydrophobic polymers of said amphiphilic copolymers, so as to be in the interior of said nanoparticle.

14. The amphiphilic copolymer of claim 1, further comprising one or more selectivity-providing molecules bonded to said amphiphilic copolymer to enable a nanocarrier formed by self-assembly of said amphiphilic copolymer to target the affected tissues.

15. The nanocarrier of claim 13, further comprising a water-insoluble or hydrophobic bioactive substance encapsulated in said nanoparticle.

16. The nanocarrier of claim 15, wherein said nanocarriers carry a mixture of said drug molecules and said water-insoluble or hydrophobic bioactive substances.

17. The nanocarrier of claim 15, wherein said water-insoluble or hydrophobic bioactive substance comprises SN-38, CPT-11 (Irinotecan), Camptothecin (CPT) or derivatives thereof, or water-insoluble or hydrophobic bioactive agents.

18. The nanocarrier of claim 13, wherein routes of administration of said nanocarriers comprise topical routes, enteral routes, or parenteral routes, wherein said topical routes comprise inhalational (pneumonial) or nasal, and said enteral routes comprise oral or rectal, wherein said parenteral routes comprise injection, infusion, transdermal or transmucosal.

19. The nanocarrier of claim 13, wherein said nanoparticle has a diameter of about 20-800 nm.

20. A fabrication method for synthesizing comb-like amphiphilic copolymers, comprising:
synthesizing an initiator, wherein said initiator comprises a drug molecule and one or two macromolecules of a hydrophobic polymer, by bonding said hydrophobic polymer to said drug molecule; and
grafting said initiators onto a pre-polymerized hydrophilic segment to form said comb-like amphiphilic copolymer.
